(19) United States
(12) Patent Application Publication
(10) Pub. No.: US 2026/0232612 A1
(43) Pub. Date: Aug. 13, 2026

(54) DIETARY SUPPLEMENT FOR MANAGING STRESS-INDUCED INFLAMMATORY SKIN DISORDERS

(71) Applicant: CODEX LABS CORPORATION, San Jose, CA (US)

(72) Inventors: Jessica MALOH, San Jose, CA (US); Barbara A. PALDUS, San Jose, CA (US); Raja SIVAMANI, Sacramento, CA (US)

(21) Appl. No.: 19/156,988

(22) PCT Filed: Feb. 16, 2024
(86) PCT No.: PCT/US2024/016121

§ 371 Date: Aug. 15, 2025

Related U.S. Application Data
(60) Provisional application No. 63/485,365, filed on Feb. 16, 2023.

(51) Int. Cl. A61K 31/197; A61K 9/00; A61K 31/047; A61K 31/07; A61K 31/10; A61K 31/12; A61K 31/19; A61K 31/192; A61K 31/194; A61K 31/198; A61K 31/202; A61K 31/327; A61K 31/357; A61K 31/519; A61K 31/60; A61K 31/7048; A61K 31/7056; A61K 33/04; A61K 36/28; A61K 36/29; A61K 36/53; A61K 36/81; A61K 36/9066; A61P 17/10

(52) U.S. Cl. CPC A61K 31/197; A61K 9/0053; A61K 31/047; A61K 31/07; A61K 31/10; A61K 31/12; A61K 31/19; A61K 31/192; A61K 31/194; A61K 31/198; A61K 31/202; A61K 31/327; A61K 31/357; A61K 31/519; A61K 31/60; A61K 31/7048; A61K 31/7056; A61K 33/04; A61K 36/28; A61K 36/29; A61K 36/53; A61K 36/81; A61K 36/9066; A61P 17/10

(57) ABSTRACT

The present disclosure relates to compositions and methods useful for dietary augmentation to provide therapeutic intervention for the treatment and management of acne-prone skin. According to one embodiment of the present disclosure, there is provided an orally administrable composition containing a therapeutically effective amount of: (a) pantothenic acid; (b) folic acid; (c) myo-inositol; (d) methylsulfonylmethane (MSM); (e) holy basil extract; (f) optionally, turmeric root extract; (g) barberry root extract; (h) L-theanine; and (i) milk thistle extract.

### FIELD OF THE INVENTION

The present disclosure relates to orally administered supplement compositions and methods of treating, ameliorating, and managing inflammatory skin disorders. More particularly, the disclosure relates to administering a therapeutically effective amount of a dietary supplement capable of inhibiting the release of inflammation-triggering hormones associated with sex hormone imbalance and psychological stress.

### BACKGROUND OF THE INVENTION

Advances in technology in the last century have brought benefits to society but have resulted in a greater prevalence of stress in the daily lives of people at all levels of society. Our stress response mechanisms have not adapted at the same pace as advancing technology. The effect of stress on health and well-being is well documented in “Why Zebra's Don't Get Ulcers—An Updated Guide to Stress, Stress Related Diseases and Coping” by Robert M. Sapolsky, ISBN 0-7167-3210-6, Chapter 1, (5th Edition 2000) and in “The Concepts of Stress and Stress System Disorders-Overview of Physical and Behavioral Homeostasis”, JAMA, Mar. 4, 1992, Vol. 267, No. 9. For example, it is known that chronic stress (an accumulation of acute stresses) can contribute to the development or aggravation of many conditions including immunosuppression and vulnerability to infectious diseases, gastric conditions, sleep problems, depression, premature birth in expectant mothers, low birth weight, degeneration of brain neurons leading to memory and learning problems, elevated blood pressure, heart complications and stroke due to elevated blood lipid levels and other health complications.

While stress has a major effect on systemic health conditions, it also has been proposed to play a role in skin health and appearance. Skin diseases such as psoriasis, atopic dermatitis, itch, uneven skin tone, poor wound repair have all been proposed to be linked to stress in some way. Some relevant references include Buske-Kirschbaum et al., Psychosomatic Medicine (1997), 59:419-426; Denda et al., Am. J. Physiol. Regulatory Integrative Comp. Physiol. 278: R367-R372, (2000); Dhabhar et al., Proc. Natl. Acad. Sci., 1999, 96:1059-1064; Kabat-Zinn et al., Psychosomatic Medicine (1998) 60:625-632; Tausk et al., Arch. Dermatol., 2001 137:78-82; Kiecolt-Glaser et al., Journal Behavioral Medicine 1986 9:5-21, Garg et al., Arch. Dermatol. 2001 137:53-59; Slominski et al., Physiological Reviews, 2000 80:979-1020; Panconesiet et al., Psychodermatology, 1996:399-421; Scholzen, et al., Exp. Dermatol. 1998 81-96, the disclosures of which are hereby incorporated by reference.

When the human mind or body are subjected to stress, the brain secretes hormones that induce the release of cortisol, a stress hormone. Skin has receptors for this stress hormone and their activation results in various changes to the skin including increased inflammation, impaired wound healing, excess sebum production, and impaired resistance to infection. These changes, in turn, can trigger various skin conditions such as acne, hair loss, hair thinning, eczema, psoriasis, and rosacea. These changes in our brain and body chemistry trigger not only skin changes, but behavioral responses as well. Individuals experiencing stress tend to touch their face because they are nervous, thereby exposing it to any pathogenic microorganisms present on their hands. Hence, if an individual naturally suffers from acne-prone skin, introduction of such microorganisms via touching can further exacerbate their acne. Stress also tends to negatively impact a person's sleeping, eating, exercising, and skin cleansing/hygiene habits which can further negatively impact their skin.

Stress, which is present among both genders and all age groups, but clearly present among the teen-age and 20-30-year-old populations, also can exacerbate and lead to acne problems via a number of different pathways. For example, chronic stress can lead to a reduction in the overall immune system, which would affect the skin's ability to fight off the C. acnes bacterium.

Hormones may act as a specific trigger for acne due to hormonal (androgen) imbalances in the body which, in turn, may cause an individual to feel stressed about their appearance triggering the release of the stress hormone cortisol. The biological domino effect of sex hormone imbalance plus stress hormone release oftentimes leads to acne-forming inflammation. It typically appears on the face, neck, and upper back and is characterized by pimples, blackheads, and whiteheads. Hormonal acne is most common in women, particularly during puberty, menstruation, pregnancy, and menopause.

Acne vulgaris (acne) is a multifactorial disease that manifests in areas rich in sebaceous glands such as the face and the trunk. Acne is a skin condition that affects over 85% of all people. It is one of the most common dermatoses affecting both men and women. More particularly, acne is a disease of the pilosebaceous gland. The acne lesions can be inflammatory (papules and pustules) and/or non-inflammatory (open and closed comedones). The following four pathogenic factors play a determining role in the formation of acne: overproduction of sebum (seborrhea), irregular desquamation, bacterial colonization, and inflammation.

These four primary factors can be affected to some degree by varying hormone levels in people. One of the major reasons why acne is prevalent among the teen-age populations is the dramatic increase in androgen output amongst that population which leads to increase sebum production. In addition, as hormone levels change in females during different times of their menstrual cycle, there is also a potential correlation with onset of acne lesions.

Recent studies have shown that the pathogenesis of acne implicates chronic inflammation at the cellular level. It has also been established that chronic inflammation causes large quantities of free radicals to be produced. These free radicals, in turn, oxidize sebum present in hair follicles, thereby decreasing its oxygen content. Cutibacterium acnes (C. acnes) is known to thrive in a high lipid environment causing it to proliferate in pores/hair follicles. Initially, this oftentimes leads to the formation of comedones (non-inflammatory acne). However, once the bacterium colonizes and overgrows in the hair follicle it triggers an inflammatory cascade in the skin that eventually leads to the formation of inflamed lesions on the skin's surface (ex. pimples). In short, chronic inflammation causes sebum oxidation; the resulting bacterial overgrowth triggers a localized inflammatory cascade in skin; the cascade exacerbates excess sebum production at the location of the infection; as well as bacterial proliferation, ultimately leading to inflammatory acne formation.

Polycystic ovary syndrome (PCOS) is a hormonal disorder that affects many women of reproductive age. It is characterized by elevated levels of androgens, like testosterone, or a higher-than-average sensitivity to progesterone that can lead to the development of multiple cysts on the ovaries and may interfere with the production of estrogen and progesterone. The hormonal imbalances associated with PCOS can lead to a range of symptoms including irregular menstrual cycles, excess hair growth, weight gain, and acne, any one or all of which can cause a person to experience psychological stress regarding their appearance which can lead to chronic inflammation/acne.

It should also be noted that psychological stress alone can cause a hormonal imbalance in the body which can lead to various inflammation-induced skin conditions. Psychological stress is any uncomfortable emotional experience accompanied by predictable biochemical, physiological, and/or behavioral changes. Psychological stress describes what people feel when they are under mental, physical, or emotional pressure. Psychological stress may aggravate natural falls in estrogen during the menstrual cycle and reduce peak levels. In men, psychological stress can lead to a reduction in testosterone levels resulting in an inflammation-triggering hormonal imbalance.

In addition, when the psychological stress hormone cortisol is released into the bloodstream, its job is to get our bodies out of stressful circumstances through a series of biological mechanisms which include: raising blood glucose levels by releasing cellular stores of glucose while triggering additional production by the liver to help fuel and power the body when it's in “fight or flight” mode; slowing down some physical biological processes such as the operation of the immune and/or digestive systems so that our body can focus on the immediate threat it's facing; and, together with adrenaline, raising our heart rate and blood pressure to more quickly deliver much needed oxygen and nutrients to the body to help repel the stress-inducing threat.

In short, cortisol is a good and necessary biological messenger that helps an individual to deal with and escape the danger they are facing, to return to a state of wellbeing as quickly as possible. If, however, cortisol is constantly being summoned by the body due to chronic stress, it can lead to insomnia, headaches, anxiety, depression, heart and memory problems, weight gain, and trigger acne flare-ups.

In addition to stress, chronic inflammation can also be caused by poor diet (food intolerances and gastrointestinal problems), environmental factors and underlying health disorders like auto-immune dysfunction. By lowering inflammation throughout the body, it is possible to decrease sebum oxidation in the skin's pores/hair follicles, thereby making the environment surrounding the hair follicle less inflammatory and in turn reduce the likelihood of non-inflammatory and inflammatory acne-formation.

Research has also shown that acne sufferers also experience greater oxidative stress compared to those with healthy skin. Because inflammation is one of the triggers that causes oxidative stress in skin through the creation of free radicals, the ability to neutralize these free radicals is highly desirable when it comes to ameliorating inflammatory skin disorders.

The correlation between a person's diet and its effect on inflammation is also well established. Numerous serious diseases such as cancer, heart disease, diabetes, arthritis, depression, and Alzheimer's have all been linked to chronic inflammation. Examples of pro-inflammatory foods include fried foods, sodas, refined carbohydrates, and red meat. Unhealthy fats, sugar, and refined carbohydrates such as those found in processed foods are all triggers of chronic inflammation. While avoidance of all pro-inflammatory foods is highly recommended, the reality is that adherence to such a strategy is easier said than done since many foods enjoyed by individuals contain one or more of these chronic inflammation triggers.

Although there are over the counter commercially available anti-acne agents for topical use (such as salicylic acid, sulfur, and benzoyl peroxide), as well as cosmetic formulations (using lactic acid, glycolic acid, pyruvic acid, urea, resorcinol, N-acetylcysteine, retinoic acid, benzoyl peroxide, octopirox, triclosan, azelaic acid, phenoxyethanol, phenoxy propanol, flavonoids, derivatives thereof, and combinations thereof) they tend to be slow acting and may have negative side effects on skin such as irritation and dryness.

In addition to the products that are available over the counter, there are also various pharmaceutical treatments that have been developed for the treatment of acne. These include, but are not limited to, both topical and oral agents, for example isotretinoin and tretinoin, adapalene, tazarotene, azelaic acid, minocycline, doxycycline, sarecycline, erythromycin, clindamycin, cyproterone acetate, spironolactone, drospirenone, flutamide, vitamins such as zinc and nicotinamide/niacinamide, which can also be used in combination with over-the-counter anti-acne agents. However, there is a continuing desire to improve the user's quality of life by reducing their reliance on these pharmaceutical compounds as they can produce serious side effects such as mood effects, joint pains, pseudotumor cerebri, development of eczema, gastrointestinal upset, and/or antibiotic resistance.

Birth control pills represent another commonly used acne treatment, particularly for hormonal acne. Androgens are hormones produced in low levels by a woman's ovaries, testes of the male, and adrenal glands. When overproduced, however, they can lead to excess sebum being produced by the body, resulting in clogged pores, inflammation, and acne formation. Birth control pills that contain both estrogen and progesterone regulate the balance of androgens in the body, resulting in less sebum production and a reduction in acne. On the other hand, progesterone-only forms of birth control such subcutaneous implants and oral pills/tablets, may further exacerbate acne, necessitating an anti-androgen-based approach to therapy.

The prominent role played by inflammation in the acne-formation process is well established. Because stress, hormones, and one's diet are considered triggers of acne-causing inflammation, it would be desirable to have an auxiliary acne treatment asset that focuses on mitigating the acne-forming effects of these triggers in a way which current treatment options do not. The use of such an auxiliary treatment asset in combination with currently available over the counter and/or pharmaceutical treatments would yield enhanced acne treatment and management results.

As a result, there remains a need for the development of new therapeutic acne treatment assets for treating and managing acne-prone skin with little to no negative side effects. Such an approach would not only help balance sex hormones and reduce chronic inflammation, but also help improve mental health and reduce psychological stress hormone levels.

### SUMMARY OF THE INVENTION

The present disclosure relates to compositions and methods useful for dietary augmentation to provide therapeutic intervention for the treatment and management of acne-prone skin. According to one embodiment of the present disclosure, there is provided an orally administrable composition containing a therapeutically effective amount of: (a) pantothenic acid; (b) folic acid; (c) myo-inositol; (d) methylsulfonylmethane (MSM); (e) holy basil extract; (f) turmeric root extract; (g) barberry root extract; (h) L-theanine; (i) milk thistle extract; and (j) optionally, ashwagandha. In some embodiments, components (e), (f), (h), (i), and (j) of the composition are optional.

According to another embodiment of the disclosure, the composition contains a therapeutically effective amount of: (a) about 50 mg of pantothenic acid; (b) about 400 mcg of folic acid; (c) about 200 mg of myo-inositol; (d) about 1000 mg of methylsulfonylmethane; (e) optionally, about 500 mg of holy basil extract; (f) optionally, about 500 mg of turmeric root extract; (g) about 300 mg of barberry root extract; (h) optionally, about 200 mg of L-theanine; and (i) optionally, about 180 mg of milk thistle extract, all weights based on the total dry weight of the composition.

According to another embodiment of the disclosure, the composition contains a therapeutically effective amount of: (a) about 50 mg of pantothenic acid; (b) about 400 mcg of folic acid; (c) about 2000 mg of myo-inositol; (d) about 1000 mg of methylsulfonylmethane; (e) about 500 mg of holy basil extract; (f) about 500 mg of turmeric root extract; (g) about 300 mg of barberry root extract; (h) about 200 mg of L-theanine; (i) about 140 mg of silymarin (extracted from milk thistle); and (j) optionally, about 300 mg of ashwagandha, all weights based on the total dry weight of the composition. In some embodiments, components (e), (f), (h), (i), and (j) of the compositions are optional.

According to another embodiment, there is provided a method of treating acne-prone skin comprising orally administering to a human subject in need thereof, a therapeutically effective oral dose of a composition containing: (a) pantothenic acid; (b) folic acid; (c) myo-inositol; (d) methylsulfonylmethane; (e) holy basil extract; (f) turmeric root extract; (g) barberry root extract; (h) L-theanine; (i) milk thistle extract; and (j) optionally, ashwagandha. In some embodiments, components (e), (f), (h), (i), and (j) of the compositions are optional.

In yet another embodiment, there is provided a method of treating acne-prone skin comprising orally administering to a human subject in need thereof, a therapeutically effective oral dose of a composition containing: (a) about 50 mg of pantothenic acid; (b) about 400 mcg of folic acid; (c) about 200 mg of myo-inositol; (d) about 1000 mg of methylsulfonylmethane; (e) optionally, about 500 mg of holy basil extract; (f) optionally, about 500 mg of turmeric root extract; (g) about 300 mg of barberry root extract; (h) optionally, about 200 mg of L-theanine; and (i) optionally, about 180 mg of milk thistle extract, all weights based on the total dry weight of the composition.

In yet another embodiment, there is provided a method of treating acne-prone skin comprising orally administering to a human subject in need thereof, a therapeutically effective oral dose of a composition containing: (a) about 50 mg of pantothenic acid; (b) about 400 mcg of folic acid; (c) about 2000 mg of myo-inositol; (d) about 1000 mg of methylsulfonylmethane; (e) about 500 mg of holy basil extract; (f) about 500 mg of turmeric root extract; (g) about 300 mg of barberry root extract; (h) about 200 mg of L-theanine; (i) about 140 mg of silymarin (extracted from milk thistle); and (j) optionally, about 300 mg of ashwagandha, all weights based on the total dry weight of the composition. In some embodiments, components (e), (f), (h), (i), and (j) of the compositions are optional.

In all of these embodiments, ashwagandha could be added to provide long-term cortisol regulating capability in order to support the fast-acting effect of 1-theanine.

These and other features, aspects and advantages of the present disclosure will become evident to those skilled in the art from a reading of the present disclosure.

### DETAILED DESCRIPTION

The compositions of the present invention can comprise, consist essentially of, or consist of, the components of the present invention as well as other ingredients described herein. The term “comprising” as used herein is meant to include various optional, compatible components that can be used in the compositions of the present disclosure. The term “consisting essentially of” as used herein means that the composition or component may include additional ingredients, but only if the additional ingredients do not materially alter the basic and novel characteristics of the compositions or methods.

As used herein, the words “preferred”, “preferably” and variants thereof refer to embodiments of the disclosure that afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the disclosure.

Numerical ranges as used herein are intended to include every number and subset of numbers contained within that range, whether specifically disclosed or not. Further, these numerical ranges should be construed as providing support for a claim directed to any number or subset of numbers within that range.

All percentages, parts, proportions, and ratios as used herein are by weight of the total composition, unless otherwise specified. All such weights as they pertain to listed ingredients are based on the active level.

The phrase “therapeutically effective amount” as used herein refers to an amount that, when administered to a subject suffering from acne, exhibits an effect of improving, treating, preventing, or inhibiting acne.

As used herein, “treatment” or “treating” means the alleviation, prophylaxis or reversal of a condition, a disease, or a disorder, or at least a discernible symptom thereof. In one embodiment, “treatment” or “treating” refers to a mitigation, prophylaxis, or reversal of at least one measurable physical parameter related to the condition, disease, or disorder being treated, not necessarily discernible in or by the individual being treated. In yet another embodiment, “treatment” or “treating” refers to inhibiting or slowing the progression of a condition, a disease, or a disorder, either physically, e.g., stabilization of a physiologically discernible symptom, e.g., stabilization of a physical parameter, or both. In yet another embodiment, “treatment” or “treating” refers to delaying the onset of a condition, disease, or disorder.

As used herein, “acne” refers to disorders resulting from the actions of hormones and other substances in the sebaceous glands and hair follicles, which typically lead to clogging of pores and the formation of inflammatory and non-inflammatory skin lesions. Specifically, it refers to blemishes, lesions or pimples, pre-emerging pimples, blackheads and/or whiteheads. As used herein, a “pre-emergent pimple” is an inflamed follicle that is not visually apparent on the surface of the skin to the naked eye (e.g., as a lesion).

As used herein, the term “inflammatory skin disorders” refers to skin conditions associated with inflammation including, for example, acne, eczema, psoriasis, and seborrheic dermatitis.

The inventors have surprisingly and unexpectedly discovered that the use of dietary supplement compositions in accordance with the present disclosure effectively ameliorate and manage inflammatory skin disorders, especially acne, based on their ability to inhibit the release of inflammation-triggering hormones associated with hormonal imbalance and/or stress, particularly psychological stress. It was also surprisingly discovered that the compositions alone, and when combined with an over the counter (OTC) and/or pharmaceutical treatment composition used for treating inflammatory skin disorders such as acne, yields synergistic results in terms of amelioration and management of inflammatory skin disorders such as acne.

Accordingly, an object of the present disclosure is to provide compositions and methods that can be used as auxiliary treatment assets for inflammatory skin disorders triggered by stress, hormones, and diet including, but not limited to, acne, psoriasis, eczema, and rosacea. According to one embodiment of the present disclosure, there is provided an orally administrable composition containing a therapeutically effective amount of: (a) pantothenic acid; (b) folic acid; (c) myo-inositol; (d) methylsulfonylmethane; (e) optionally, holy basil extract; (f) optionally, turmeric root extract; (g) barberry root extract; (h) L-theanine; (i) optionally, milk thistle extract and (j) optionally, ashwagandha. In some embodiments, component (h) of the composition is optional.

Pantothenic acid is a water-soluble vitamin, also known as vitamin B5, is used to make coenzyme A (CoA), a chemical compound that helps enzymes to build and break down fatty acids and perform other metabolic functions. Pantothenic acid is found in a large range of foods, such as fish, meat, eggs, milk products and vegetables. The pantothenic acid may be employed in an amount of from about 30 to about 75 mg, such as from about 40 to about 60 mg, and preferably about 50 mg, all weights based on the total dry weight of the composition.

Folic acid is a form of vitamin B9 and is found in many foods such as leafy vegetables, legumes, mushrooms, and orange juice. It is typically used for conditions such as depression, stroke, and decline in memory. It has also been shown to improve insulin resistance and decrease testosterone levels. The folic acid may be employed in an amount of from about 200 to about 600 mcg, such as from about 300 to about 500 mcg, and preferably about 400 mcg, all weights based on the total dry weight of the composition.

Inositol (myo-inositol) is a carbocyclic sugar abundant in the brain and other mammalian tissue. Its function is to mediate cell signal transduction in response to a variety of hormones, neurotransmitters, and growth factors. It is promoted as a dietary supplement capable of managing polycystic ovary syndrome (PCOS). It is also used to reduce insulin resistance which is implicated in the production of MCP1 in adipocytes. MCP1 is believed to play a role in the recruitment of monocytes and activates pro-inflammatory M1 macrophages as evidenced by inflammatory gene expression. In addition, the consistent administration of inositol has been found to significantly reduce plasma LH, testosterone, free testosterone, insulin, and HOMA index. The inositol may be employed in amounts ranging from about 1000 to about 3000 mg, such as from about 1500 to about 2500 mg, and particularly about 2000 mg, all weights based on the total dry weight of the composition.

Methylsulfonylmethane (MSM) is a naturally occurring organosulfur compound used as a popular dietary supplement for reducing inflammation, joint and muscle pain, oxidative stress, and antioxidant capacity. Because it's a Generally Recognized As Safe (GRAS) approved substance, it is well tolerated by most individuals. MSM may be employed in amounts ranging from about 500 to about 2000 mg, such as from about 750 to about 1500 mg, and preferably about 1000 mg, all weights based on the total dry weight of the composition.

Holy Basil (Tulsi) extract is an herb commonly used in Ayurveda. Studies have suggested that it can be used to relieve anxiety, stress, and depression. It may also be associated with a decrease in glucose levels, inflammation, and joint pain, as well as help enhance the body's immune system. Holy Basil extract may be employed in amounts ranging from about 250 to about 1000 mg, such as from about 400 to about 750 mg, and preferably about 500 mg, all weights based on the total dry weight of the composition.

Turmeric root extract is an herb also commonly used in Ayurveda due to it having curcuminoids as an active ingredient and has been promoted as a dietary supplement for a variety of conditions including arthritis, digestive disorders, and depression, to name a few. It is believed to possess anti-inflammatory properties and has been studied on various skin conditions such as psoriasis, itchy skin, oral lichen planus, facial redness, and certain forms of skin cancer. Turmeric root extract may be employed in amounts ranging from about 250 to about 1000 mg, such as from about 400 to about 750 mg, and preferably about 500 mg, all weights based on the total dry weight of the composition. The quantity of curcuminoids employed can range from about 25 to about 100 mg, such as from about 40 to about 75 mg, and preferably about 50 mg, based on the total dry weight of the composition.

Barberry extract is yet another herb used in Ayurveda as a dietary supplement for its anti-inflammatory and antioxidant properties and is believed to promote vigor and a sense of well-being. Scientifically, berberine and other compounds in barberries not only have anti-inflammatory and antibacterial properties, but the anti-lipogenic effect of barberry on the sebaceous glands may further support its effectiveness as an anti-acne agent. Whereas supplements typically involve the use of extracts derived from barberry berries, the present disclosure is preferential to the extract derived from the root portion. The Barberry root extract may be employed in amounts ranging from about 150 to about 600 mg, such as from about 200 to about 400 mg, and preferably about 300 mg, all weights based on the total dry weight of the composition.

L-theanine is a unique water-soluble non-protein amino acid found in green/black tea and mushrooms, that is believed to promote relaxation. It is believed to be able to modulate aspects of brain function resulting in a significant decrease in stress- and anxiety-related symptoms. When taken by mouth, L-theanine is considered safe when used in the short-term (about 8 weeks) in doses of up to about 900 mg per day. L-theanine may be employed in amounts ranging from about 100 to about 400 mg, such as from about 150 to about 300 mg, and preferably about 200 mg, all weights based on the total dry weight of the composition.

Milk thistle extract is derived from the milk thistle plant named for the white veins on its large prickly leaves. One of its primary active ingredients, silymarin, is extracted from the plant's seeds. It is used as a dietary supplement for its antioxidant, liver-supporting, and anti-inflammatory properties. It is thought to possess beneficial properties similar to that of doxycycline when it comes to acne treatment. Milk thistle extract may be employed in amounts ranging from about 100 to about 300 mg, such as from about 150 to about 200 mg, and preferably about 180 mg, all weights based on the total dry weight of the composition. In terms of its active ingredient silymarin, it may be employed in amounts ranging from about 75 to about 250 mg, such as from about 100 to about 200 mg, and preferably about 140 mg, all weights based on the total dry weight of the composition.

Yet another ingredient which may also be employed by the invention of the present disclosure for its stress-reducing properties is ashwagandha. It is an herbal shrub whose roots and berries have been used for their medicinal properties. The addition of ashwagandha, a long-term cortisol regulator, together with 1-theanine, a short-term cortisol regulator, is believed to synergistically aid in cortisol regulation/resilience by the body. Ashwagandha may be employed in amounts ranging from about 100 to about 1000 mg, such as from about 200 to about 600 mg, and particularly about 300 mg, all weights based on the dry weight of the composition.

According to one embodiment of the present disclosure, there is provided an orally administrable composition containing a therapeutically effective amount of: (a) pantothenic acid; (b) folic acid; (c) myo-inositol; (d) methylsulfonylmethane (MSM); (e) holy basil extract; (f) turmeric root extract; (g) barberry root extract; (h) L-theanine; (i) milk thistle extract; and (j) optionally, ashwagandha.

According to an embodiment of the disclosure, the composition contains a therapeutically effective amount of: (a) from about 30 to about 75 mg of pantothenic acid; (b) from about 200 to about 60 mcg of folic acid; (c) from about 1000 to about 3000 mg of myo-inositol; (d) from about 500 to about 2000 mg of methylsulfonylmethane; (e) from about 250 to about 1000 mg of holy basil extract; (f) from about 250 to about 1000 mg of turmeric root extract having from about 25 to about 100 curcuminoids; (g) from about 150 to about 600 mg of barberry root extract; (h) from about 100 to about 400 mg of L-theanine; (i) from about 100 to about 300 mg of milk thistle extract having from about 75 to about 250 mg of silymarin; and (j) optionally, from about 100 to about 1000 mg of ashwagandha, all weights based on the total dry weight of the composition.

According to another embodiment of the disclosure, the composition contains a therapeutically effective amount of: (a) from about 40 to about 60 mg of pantothenic acid; (b) from about 300 to about 500 mcg of folic acid; (c) from about 1500 to about 2500 mg of myo-inositol; (d) from about 750 to about 1500 mg of methylsulfonylmethane; (e) from about 400 to about 750 mg of holy basil extract; (f) from about 400 to about 750 mg of turmeric root extract having from about 40 to about 75 curcuminoids; (g) from about 200 to about 400 mg of barberry root extract; (h) from about 150 to about 300 mg of L-theanine; and (i) from about 150 to about 200 mg of milk thistle extract having from about 100 to about 200 mg of silymarin; and (j) optionally, from about 200 to about 600 mg of ashwagandha, all weights based on the total dry weight of the composition.

According to yet another embodiment of the present disclosure, the composition contains a therapeutically effective amount of: (a) about 50 mg of pantothenic acid; (b) about 400 mcg of folic acid; (c) about 2000 mg of inositol; (d) about 1000 mg of methylsulfonylmethane; (e) about 500 mg of holy basil extract; (f) about 500 mg of turmeric root extract having about 50 mg of curcuminoids; (g) about 300 mg of barberry root extract; (h) about 200 mg of L-theanine; and (i) about 180 mg of milk thistle extract having about 140 mg of silymarin; and (j) optionally, about 300 mg of ashwagandha, all weights based on the total dry weight of the composition. In some embodiments, components (e), (f), (h), (i), and (j) of the composition are optional.

In yet another embodiment of the present disclosure, there is provided a method of treating acne-prone skin comprising orally administering to a human subject in need thereof, a therapeutically effective oral dose of a composition containing: (a) from pantothenic acid; (b) folic acid; (c) myo-inositol; (d) methylsulfonylmethane; (e) holy basil extract; (f) turmeric root extract; (g) barberry root extract; (h) L-theanine; (i) milk thistle extract; and (j) optionally, ashwagandha.

According to one embodiment of the present disclosure, there is provided a method of treating acne-prone skin comprising orally administering to a human subject in need thereof, a therapeutically effective oral dose of a composition containing: (a) from about 30 to about 75 mg of pantothenic acid; (b) from about 200 to about 60 mcg of folic acid; (c) from about 1000 to about 3000 mg of myo-inositol; (d) from about 500 to about 2000 mg of methylsulfonylmethane; (e) from about 250 to about 1000 mg of holy basil extract; (f) from about 250 to about 1000 mg of turmeric root extract having from about 25 to about 100 curcuminoids; (g) from about 150 to about 600 mg of barberry root extract; (h) from about 100 to about 400 mg of L-theanine; (i) from about 100 to about 300 mg of milk thistle extract having from about 75 to about 250 mg of silymarin; and (j) optionally, from about 100 to about 1000 mg of ashwagandha, all weights based on the total dry weight of the composition.

According to another embodiment of the present disclosure, there is provided a method of treating acne-prone skin comprising orally administering to a human subject in need thereof, a therapeutically effective oral dose of a composition containing: (a) from about 40 to about 60 mg of pantothenic acid; (b) from about 300 to about 500 mcg of folic acid; (c) from about 1500 to about 2500 mg of myo-inositol; (d) from about 750 to about 1500 mg of methylsulfonylmethane; (e) from about 400 to about 750 mg of holy basil extract; (f) from about 400 to about 750 mg of turmeric root extract having from about 40 to about 75 curcuminoids; (g) from about 200 to about 400 mg of barberry root extract; (h) from about 150 to about 300 mg of L-theanine; (i) from about 150 to about 200 mg of milk thistle extract having from about 100 to about 200 mg of silymarin; and (j) optionally, from about 200 to about 600 mg of ashwagandha, all weights based on the total dry weight of the composition.

According to another embodiment of the present disclosure, there is provided a method of treating acne-prone skin comprising orally administering to a human subject in need thereof, a therapeutically effective oral dose of a composition containing: (a) about 50 mg of pantothenic acid; (b) about 400 mcg of folic acid; (c) about 2000 mg of inositol; (d) about 1000 mg of methylsulfonylmethane; (e) about 500 mg of holy basil extract; (f) about 500 mg of turmeric root extract having about 50 mg of curcuminoids; (g) about 300 mg of barberry root extract; (h) about 200 mg of L-theanine; (i) about 180 mg of milk thistle extract having about 140 mg of silymarin; and (j) optionally, about 300 mg of ashwagandha, all weights based on the total dry weight of the composition.

In those embodiments in which ashwagandha is employed, the short-term cortisol lowering effect of L-theanine, which acts in about 3 hours on an average individual, is believed to be synergistically supported with the long-term adaptogenic regulating ability of ashwagandha, to help the body build resilience to sudden spikes in cortisol.

Besides their direct and independent benefits, the ingredients of the dietary supplements of the invention are believed to work together synergistically in a manner that powerfully enhances each ingredient's level of efficacy, thereby further promoting their beneficial treatment effect. Without intending to be bound by theory, the inventors believe that the compositions of the present disclosure by virtue of their ability to reduce psychological stress, rebalance sex hormones (e.g. reducing excess levels of androgens and/pr testosterone), inhibit the release of pro-inflammatory hormones caused by stress and a poor diet, and provide anti-inflammatory and antioxidant properties, results in the synergistic amelioration of inflammatory skin disorders such as acne, and promotion of healthy skin.

According to yet another embodiment, the present disclosure is also directed to a method of treating acne-prone skin, comprising orally administering to a human subject in need thereof, a therapeutically effective oral dose of the above-disclosed compositions.

In another embodiment, the method can further include topically applying a therapeutically effective amount of an ancillary treatment composition containing at least one active ingredient chosen from benzoyl peroxide, sulfur, salicylic acid, azelaic acid, alpha hydroxy acids, bakuchiol, retinol, retinoids (tretinoin, tazarotene, trifarotene, adapalene), glycolic acid, clascoterone, erythromycin and clindamycin.

Acne is a chronic inflammatory condition that is estimated to affect more than 85% of the population at some point. While antibiotics are typically used for systemic therapy, they can increase a person's risk for developing drug-resistance to bacteria while shifting the gut microbiome in a negative direction. Other commonly used treatments may also be problematic due to their potential side effects. For example, spironolactone used for treating hormonal acne has potential side effects such as causing low blood pressure, buildup of potassium in the blood, headaches, breast tenderness, and irregular menstruation. Isotretinoin, another widely used treatment option, has been found to inhibit sebaceous gland function and keratinization at pharmacologic doses. This drug has been observed to reduce both sebaceous gland size and sebum production. Since sebum produced by the sebaceous gland represents the body's naturally produced skin moisturizer, these side effects can negatively affect the proper functioning of the skin barrier which, in turn, can have a significant impact on skin health and appearance. Additional, more serious, side effects include headaches associated with pseudotumor cerebri, joint pains, decreased low level light vision, liver inflammation, and development of eczema.

The following examples as set forth herein are intended for illustrative purposes only and are not intended to limit the scope of the disclosure in any way, as many variations thereof are possible without departing from the spirit and scope of the disclosure. In the example, all concentrations are listed as dry weight percent, unless otherwise specified.

### Example 1

| | | |
|---|---|---|
| Ingredient | % dry wt/wt | % dry wt/wt |

| | | | |
|---|---|---|---|
| Pantothenic acid | 50 | mg | |
| Folic acid | 400 | mcg | |
| Inositol | 2000 | mg | |
| MSM | 1000 | mg | |
| Holy Basil extract | 500 | mg | |
| Turmeric root extract | 500 | mg | |
| curcuminoids | | | 50 mg |
| Barberry root extract | 300 | mg | |
| L-theanine | 200 | mg | |
| Milk thistle extract | 180 | mg | |
| silymarin | | | 140 mg |

The composition of Example 1 was evaluated to assess its impact on individuals suffering from acne. A clinical study was performed on a group of chronic acne sufferers, aged 12 to 45, to determine what, if any, benefit could be realized by taking the supplement of Example 1. Each participant was asked to take a daily dose of the composition of Example 1 over a period of 8 weeks. The participants were classified as having mild to moderate noncystic acne, based on the investigator's global assessment, and had to have at least 10 inflammatory lesions, and at least 5 non-inflammatory lesions in order to qualify for the study. The main testing method for judging efficacy was inflammatory (papules and pustules) and non-inflammatory (open and closed comendones) lesion counting. The results of the study are found in Table 1 below.

TABLE 1

| | Non-inflammatory | | Inflammatory | |
|---|---|---|---|---|
| | lesion change | | lesion change | |
| | from baseline (%) | | from baseline (%) | |
| | Week 4 | Week 8 | Week 4 | Week 8 |
| Average | −29.8% | −63% | −55.6 | −58.7 |
| Improvement | | | | |
| SEM | 7.00% | 5.20% | 5.70% | 9.40% |
| p Value | 0.0011 | 0.00000004 | 0.0000004 | 0.000042 |

As can be seen from the results in Table 1, chronic acne sufferers realized both a statistically and clinically significant reduction in both inflammatory lesions (papules and pustules) and non-inflammatory lesions (open and closed comedones).

What is claimed is:

1. An orally administrable composition comprising a therapeutically effective amount of: (a) pantothenic acid; (b) folic acid; (c) inositol; (d) methylsulfonylmethane; (e) holy basil extract; (f) turmeric root extract; (g) barberry root extract; (h) L-theanine; (i) milk thistle extract; and (j) optionally, ashwagandha.

2. The composition of claim 1, wherein (a) is employed in an amount of from about 30 to about 75 mg; (b) is employed in an amount of from about 200 to about 600 mcg; (c) is employed in an amount of from about 1000 to about 3000 mg; (d) is employed in an amount of from about 500 to about 2000 mg; (e) is employed in an amount of from 250 to about 1000 mg; (f) is employed in an amount of from about 250 to about 1000 mg, and contains curcuminoids in an amount of from about 25 to about 100 mg; (g) is employed in an amount of about 150 to about 600 mg; (h) is employed in an amount of from about 100 to about 400 mg; and (i) is employed in an amount of from about 100 to about 300 mg and contains silymarin in an amount of from about 75 to about 250 mg; and (j) is optionally employed in an amount of from about 100 to about 1000 mg of ashwagandha, all weights based on the total dry weight of the composition.

3. The composition of claim 1, wherein (a) is employed in an amount of from about 40 to about 60 mg; (b) is employed in an amount of from about 300 to about 500 mcg; (c) is employed in an amount of from about 1500 to about 2500 mg; (d) is employed in an amount of from about 750 to about 1500 mg; (e) is employed in an amount of from 400 to about 750 mg; (f) is employed in an amount of from about 400 to about 750 mg and contains curcuminoids in an amount of from about 40 to about 75 mg; (g) is employed in an amount of about 200 to about 400 mg; (h) is employed in an amount of from about 150 to about 300 mg; (i) is employed in an amount of from about 150 to about 200 mg and contains silymarin in an amount of from about 100 to about 200 mg; and (j) is optionally employed in an amount of from about 200 to about 600 mg, all weights based on the total dry weight of the composition.

4. The composition of claim 1, wherein (a) is employed in an amount of about 50 mg; (b) is employed in an amount of about 400 mcg; (c) is employed in an amount of about 2000 mg; (d) is employed in an amount of about 1000 mg; (e) is employed in an amount of about 500 mg; (f) is employed in an amount of about 500 mg and contains curcuminoids in an amount of about 50 mg; (g) is employed in an amount of about 300 mg; (h) is employed in an amount of about 200 mg; (i) is employed in an amount of about 180 mg and contain silymarin in an amount of about 140 mg; and (j) is optionally employed in an amount of about 300 mg, all weights based on the total dry weight of the composition.

5. A method of treating an inflammatory skin disorder comprising orally administering the composition of claim 1 to a human subject in need thereof.

6. A method of treating an inflammatory skin disorder comprising orally administering the composition of claim 2.

7. A method of treating an inflammatory skin disorder comprising orally administering the composition of claim 3.

8. A method of treating an inflammatory skin disorder comprising orally administering the composition of claim 4.

9. The method of claim 5, wherein the composition is orally administered once daily.

10. The method of claim 9, wherein the composition is orally administered once daily over a period of about 8 weeks.

11. The method of claim 5, wherein the inflammatory skin disorder is chosen from acne vulgaris, psoriasis, eczema, and rosacea.

12. The method of claim 5, wherein the inflammatory skin disorder is acne vulgaris.

13. The method of claim 5, further comprising topically applying a therapeutically effective amount of an ancillary treatment composition containing at least one active ingredient selected from benzoyl peroxide, adapalene, sulfur, salicylic acid, azelaic acid, alpha hydroxy acids, retinol, retinoids, glycolic acid, tretinoin, erythromycin, clindamycin, or combinations thereof.
